(19) United States
(12) Patent Application Publication
(10) Pub. No.: US 2025/0315946 A1
(43) Pub. Date: Oct. 9, 2025

(54) METHOD FOR PROCESSING 3D IMAGING DATA AND ASSISTING WITH PROGNOSIS OF CANCER

(71) Applicants: INSTITUT NATIONAL DE LA SANTÉ ET DE LA RECHERCHE MÉDICALE, Paris (FR); INSTITUT CURIE, Paris (FR); UNIVERSITE PARIS-SACLAY, Gif-sur-Yvette (FR)

(72) Inventors: Irène BUVAT, Gif-sur-Yvette (FR); Kibrom GIRUM, Gif-sur-Yvette (FR)

(21) Appl. No.: 18/863,355

(22) PCT Filed: May 17, 2023
(86) PCT No.: PCT/EP2023/063366

§ 371 Date: Nov. 6, 2024

(30) Foreign Application Priority Data
May 19, 2022 (EP) 22305747.2

(51) Int. Cl. G06T 7/00; G06N 3/0455; G06N 3/0464; G06T 3/06; G06V 10/75; G16H 30/40; G16H 50/20

(52) U.S. Cl. CPC G06T 7/0012; G06N 3/0455; G06N 3/0464; G06T 3/06; G06V 10/758; G16H 30/40; G16H 50/20; G06T 2207/10104; G06T 2207/20084; G06T 2207/30096

(57) ABSTRACT

It is disclosed a method processing imaging data of a patient having cancer, for instance lymphoma, comprising:—Providing three-dimensional imaging data of the patient,—computing from said three-dimensional imaging data, at least one two-dimensional Maximum Intensity Projection image. corresponding to the projection of the maximum intensity of the three-dimensional imaging data along one direction onto one plane,—extracting a mask of the MIP image corresponding to cancerous lesions by application of a trained model. Using the extracted mask it is possible to compute one or more cancer prognosis indicators.

### TECHNICAL FIELD

The present disclosure relates to the field of medical imaging, more specifically to the processing of three-dimensional imaging data of patients having cancer.

### PRIOR ART

Diffuse large B-cell lymphoma (DLBCL) is the most common type of non-Hodgkin lymphoma.

In clinical practice, acquiring F-FDG PET/CT image is a standard-of-care for staging and assessing response in DLBCL patients. Positron Emitting Tomography (PET) is a technology which allows locating a radiotracer which has been previously injected in a patient. Typically chosen radiotracers, such as fluorodeoxyglucose 18F-FDG, accumulate on the regions of the body which include cells with a high metabolic activity. Such regions include brain, liver, and tumors. PET scan imaging thus allows mapping the tumors of a patient.

Moreover, once a 18F-FDG PET/CT image is acquired on the patient, this image can be processed to compute one or more biomarkers having prognostic value for the patient. It has been largely demonstrated that the total metabolically active tumor volume (TMTV) calculated from 18F-FDG PET images has prognostic value in lymphoma, and especially in DLBCL (Mikhaeel N G, Smith D, Dunn J T et al. “Combination of baseline metabolic tumour volume and early response on PET/CT improves progression-free survival prediction in DLBCL”. Eur J Nucl Med Mol Imaging. 2016;43:1209-1219). The disease dissemination, reflected by the largest distance between two lesions in the baseline whole-body 18F-FDG PET/CT image (Dmax), has also been shown to be an early prognostic factor (Cottereau A -S, Nioche C, Dirand A -S et al. 18 F-FDG PET Dissemination Features in Diffuse Large B-Cell Lymphoma Are Predictive of Outcome, J Nucl Med. 2020; 61:40-45).

TMTV and Dmax calculations require tumor volume delineation over the whole-body three-dimensional (3D) 18F-FDG PET/CT images, which is time consuming (up to 30 min per patient), prone to observer-variability and complicates the use of these quantitative features in clinical routine.

To address this problem, automated lesion segmentation approaches using convolutional neural networks (CNN) have been proposed in:
Sibille L, Seifert R, Avramovic N, et al. «18 F-FDG PET/CT Uptake Classification in Lymphoma and Lung Cancer by Using Deep Convolutional Neural Networks”. Radiology. 2020;294:445-452
Blanc-Durand P, Jegou S, Kanoun S, et al. «Fully Automatic segmentation of Diffuse Large B-cell Lymphoma lesions on 3D FDG-PET/CT for total metabolic tumour volume prediction using a convolutional neural network”. Eur J Nucl Med Mol Imaging. 2021;48:1362-1370.

These methods have shown promising results, but they require high computational resources to be developed, and tend to miss small lesions. Further, results from CNN still need to be validated and adjusted by an expert before using them for further analysis and subsequent biomarker calculation. This implies a thorough visual analysis of all 3D 18F-FDG PET/CT images and delineation of the lesions missed by the algorithm. Consequently, developing a pipeline that would fully automate the segmentation and/or speed-up this checking/adjustment process is highly desirable in clinical practice.

### SUMMARY OF THE INVENTION

The aim of the present disclosure it to address the limitations of the prior art. In particular, an aim of the invention is to provide a method for processing three-dimensional imaging data of a patient having cancer in order to delineate a lesion region that is more reliable and less computationally-intensive than state-of-the-art method, and reduces the time needed by an expert to perform post-processing validation.

Accordingly, the present disclosure relates to a method of processing imaging data of a patient having cancer, comprising:
providing three-dimensional imaging data of the patient,
computing from said three-dimensional imaging data, at least one two-dimensional Maximum Intensity Projection image, corresponding to the projection of the maximum intensity of the three-dimensional imaging data along one direction onto one plane,
extracting a mask of the MIP image corresponding to cancerous lesions by application of a trained model.

In embodiments, the three-dimensional imaging data is PET scan data.

In embodiments, the method comprises computing from the three-dimensional imaging data two Maximum Intensity Projection images corresponding to the projection of the maximum intensity of the three-dimensional imaging data onto two orthogonal planes. In this case, the model may have been previously trained by supervised learning on a database comprising a plurality of MIP images corresponding to projections of three-dimensional imaging data according to a first plane, and a plurality of MIP images corresponding to projections of three-dimensional imaging data according to a second plane, orthogonal to the first, and, for each MIP image, a corresponding mask of the image corresponding to cancerous lesions.

In embodiments, wherein the trained model is a Convolutional Neural Network comprising:
an encoder region comprising a succession of layers of decreasing resolutions,
a decoder region comprising a succession of layers of increasing resolutions, wherein a layer of the decoder region concatenates the output of the layer of the encoder region of the same resolution with the output of the layer of the decoder region of the next lower resolution,
a bottle-neck region between the encoder and decoder regions, and
a feedback linking the output of the network and the bottle-neck region.

In embodiments, the encoder, decoder and bottle-neck regions of the network comprise building blocks where each building block is a residual block comprising at least a convolutional layer and an activation layer, with a skip connection between the input of the block and the activation layer.

It is also disclosed a method for assisting with cancer prognosis comprising:
performing the method of processing imaging data according to the above description on three-dimensional imaging data of a patient to output a two-dimensional cancerous lesion mask of a MIP image computed from the three-dimensional imaging data, and
processing said cancerous lesion mask to compute at least one prognosis indicator.

In embodiments, the at least one prognosis indicator comprises an indicator of the lesion dissemination.

In embodiments, processing the cancerous lesion mask comprises computing the distance between tumor pixels belonging to the mask along two orthogonal axes of the mask and summing said dimensions.

In embodiments, at least one prognosis indicator comprises an indicator of the lesion burden.

In embodiments, processing the cancerous lesion mask comprises computing a number of pixels belonging to the lesion multiplied by the area represented by each pixel.

In embodiments, the cancer is a lymphoma, for instance a Diffuse Large B-cell Lymphoma.

It is also disclosed a computer-program product comprising code instructions for implementing the methods of processing imaging data and for assisting with cancer prognosis according to the above description, when it is executed by a processor.

It is also disclosed a non-transitory computer readable storage having stored thereon code instructions for implementing the methods of processing imaging data and for assisting with cancer prognosis according to the above description, when they are executed by a processor.

The proposed method allows automatically segmenting cancerous lesions regions from 3D imaging data such as PET imaging data, by performing said segmentation on 2D Maximum Intensity Projection (MIP) images obtained from said 3D data, using a trained model. The computational resources needed to train and execute the trained model on a 2D MIP image are very much reduced as compared to the training and execution of a model on PET imaging data, and the checking/adjustment process performed by an expert is speeded-up since the expert does not need to analyze a whole 3D PET image, but only the 2D MIP images(s).

Meanwhile, the lesion region that is extracted from the 2D MIP image can be processed to extract indicators reflecting the volume of the tumor and the tumor dissemination which are prognosis indicators that can serve as a basis to estimate the chances of survival of the patient (overall survival OS), or the chances of progression-free survival (PS).

### DESCRIPTION OF THE DRAWINGS

Other features and advantages of the invention will be apparent from the following detailed description given by way of non-limiting example, with reference to the accompanying drawings, in which:

FIG. 1 schematically represents the main steps of a method according to an embodiment,

FIG. 2 represents 18F-FDG PET MIP images and segmentation results (blue color overlapped over the PET MIP images) by experts (MIP_masks) and by the CNN for four patients: (A, B) from the REMARC patient cohort, and (C, D) from the LNH073B patient cohort.

FIG. 3 schematically represents the structure of a Convolutional Neural Network that may be used for segmenting MIP images,

FIG. 4 illustrates the computation of the lesion dissemination feature from a MIP image.

FIG. 5 displays Kaplan-Meier estimates of overall survival (OS) and progression-free survival (FPS) on the REMARC cohort according to 3D 18F-FDG PET/CT image-based features TMTV (cm3) and Dmax (cm) (A, C), and according to PET MIP image-based features (I_{B} (cm2) and I_{D} (cm)) estimated from AI (B, D).

FIG. 6 displays Kaplan-Meier estimates of overall survival (OS) and progression-free survival (FPS) on the LNH073B cohort according to 3D 18F-FDG PET/CT image-based features TMTV (cm3) and Dmax (cm) (A, C), and according to PET MIP image-based features (I_{B} (cm2) and I_{D} (cm)) estimated from AI (B, D).

FIG. 7 displays confusion matrices for classification of patients using PET features derived from the expert-delineated 3D 18F-FDG PET regions (3D-expert) and from the 2D PET MIP regions delineated by the CNN (2D-AI) on LNH073B cohort. A) Two-risk-group classification using Dmax and I_{D}, B) two-risk-group classification using TMTV and I_{B}, and C) three-risk-group classification using TMTV and Dmax (3D-expert), and I_{B} and I_{D} (CNN).

### DETAILED DESCRIPTION OF AT LEAST ONE EMBODIMENT

With reference to the drawings, a method for processing three-dimensional imaging data of a patient having cancer, and of extracting prognosis indicators therefrom, will now be described.

The method may be implemented by a computing system comprising at least one processor, which may include one or more Computer processing unit(s) CPU, and/or Graphical Processing Unit(s) GPU, and a non-transitory computer-readable medium 11 storing program code that is executable by the processor, to implement the method described below. The computing system 1 may also comprise at least one memory 12 storing a trained model configured for extracting cancer lesion region or mask from a Maximum Intensity Projection (MIP) Image obtained from three dimensional imaging data of a patient.

In embodiments, the method disclosed below may be implemented as software program by a PET/CT scanner incorporating said at least one processor, and which may also store the memory 12 for accessing the stored model. Alternatively, the memory may be remotely located and accessed via a data network, for instance a wireless network.

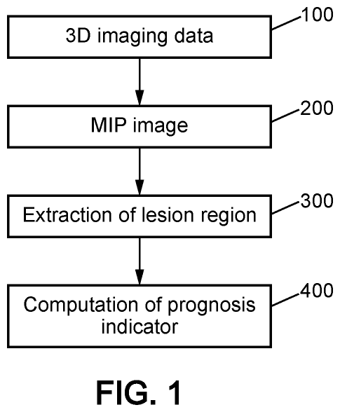

With reference to FIG. 1, the method comprises providing 100 three-dimensional PET imaging data of a patient having cancer.

The three-dimensional imaging data may be Positron Emission Tomography imaging data obtained with 18F-FDG tracer. The three-dimensional imaging data may be acquired from skull base to upper thighs of a patient, and is later denoted as whole-body imaging data. In embodiments, step 100 does not include the actual acquisition of imaging data on a patient, but may comprise recovering said data from a memory, Picture Archiving and Communication System (PACS) or network in which it is stored.

The cancer may be any type of cancer, metastatic or not, including colorectal cancer, breast cancer, lung cancer, lymphoma, in particular non-Hodgkin lymphoma, in particular Diffuse Large B-Cell Lymphoma (DLBCL).

The method then comprises computing 200 from said three-dimensional imaging data, at least one two-dimensional Maximum Intensity Projection (MIP) image, corresponding to the projection of the maximum intensity of the three-dimensional imaging data onto one plane. In other words, a MIP image is a 2D image in which each pixel value is equal to the maximum intensity of the 3D imaging data observed along a ray normal to the plane of projection.

In embodiments, the plane of projection of the MIP image may be the coronal plane, i.e. the vertical plane that partitions the body into front, and back. The plane of projection of the MIP image may also be the sagittal plane, i.e. the vertical plane that partitions the body into left and right halves.

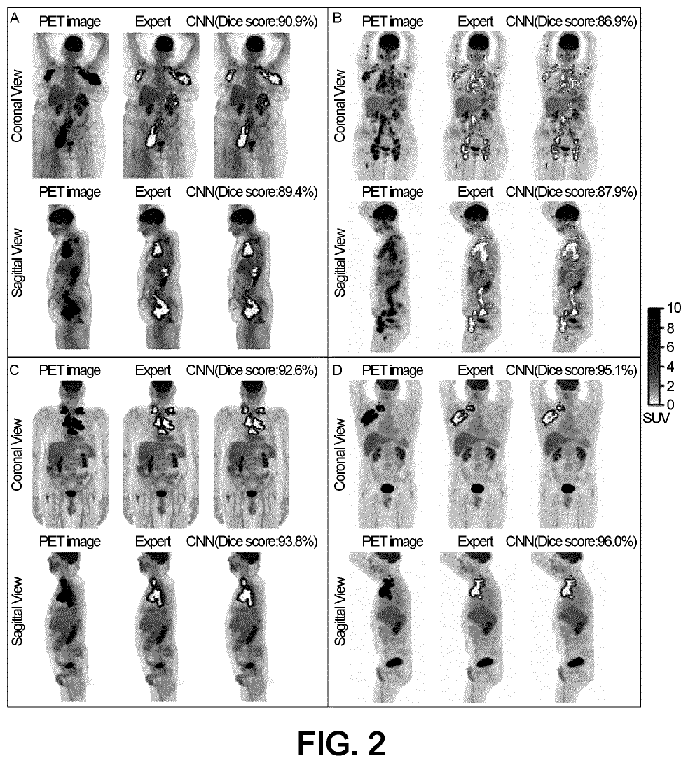

In embodiments, one, two or more MIP images are computed from the 3D imaging data, where the MIP images preferably correspond to projections of the maximum intensity of the 3D imaging data along two orthogonal planes. According to an embodiment shown in FIG. 2, step 200 may comprise computing one MIP image along the sagittal plane, and one MIP image along the coronal plane.

The method then comprises extracting 300 from said at least one 2D MIP image a mask corresponding to cancerous lesions. The mask extracted from the MIP image is a two-dimensional image, that may have the same size as the MIP image, in which the pixels corresponding to cancer lesions are set to one, and the other are set to zero.

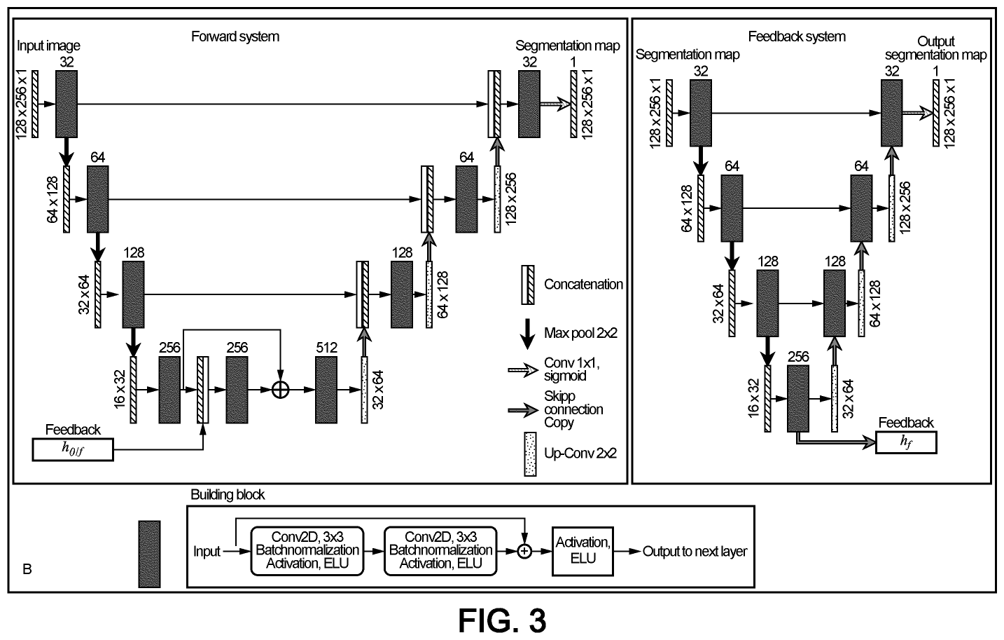

This extraction, or segmentation, is performed by a trained model that is configured to extract from 2D MIP images obtained from 3D imaging data, in particular 18F-FDG PET imaging data, a mask of the cancerous lesions. The trained model may be a Convolutional Neural Network (CNN), in particular having a U-Net architecture, In embodiments, the trained model may be the model disclosed by Kibrom Berihu Girum et al. “Learning with Context Feedback Loop for Robust Medical Image Segmentation”, in IEEE Transactions on Medical Imaging, arXiv: 2103.02844, 2021, having the structure shown in FIG. 3.

This CNN comprises a main, forward system, comprising an encoder region encoding the raw MIP input image into a feature space, a decoder region decoding the encoded features into target labels, and a bottle-neck region or processing region of the feature space. The CNN further comprises skipped connections between the encoder and decoder regions.

The encoder region comprises a succession of layers of decreasing resolution, where each layer comprises a convolutional building block discussed in more details below, and each layer except the first performs a Max Pooling on the output of the building block of the preceding layer of higher resolution.

The decoder region also comprises a convolutional building block that receives as input the output of the encoder layer of same resolution through a skip connection, concatenated with the output of an up-convolutional layer applied to the output of the building block of the preceding layer of lower resolution.

The bottle-neck region is a residual block with a skip connection between the output of the last layer of the encoder region and the input of the first layer of the decoder region.

The building block in all components of the model is a residual CNN, comprising convolutional layers and an activation layer, with a skip connection between the input of the block and the activation layer. This can ease training and facilitate information propagation from input to the output of the network architecture. In particular in the case of lymphoma, lesions can be scattered over the whole body and the choice of this building block prevents losing information in the successive convolution and pooling operations.

As shown in the left-hand part of FIG. 3, such network further comprises an external fully-connected network-based feedback system. The feedback system links the output of the CNN, i.e. the segmentation map or segmented region of the image, to the bottleneck region. As shown in the right-hand part of FIG. 3, the feedback system also has a structure of encoder-decoder, with the encoder and decoder parts being identical respectively to the encoder and decoder parts of the main forward system represented in the left-hand part of FIG. 3, but with the output of the last convolutional building block of the encoder being fed directly to the first up-convolutional layer of the decoder block. The output of the CNN is thus encoded by the feedback system into the same high-feature space as the bottle-neck region of the main forward system represented in the left-hand-part of FIG. 3.

The output h_{f} of the last convolutional building block of the encoder can be concatenated with the output of the building block of the layer of lowest resolution of the main forward system for at least one training phase of the network.

The training of such model may comprise a series of steps including:
Training the network weights of the forward system, considering raw input images and zero feedback (denoted h₀ in FIG. 3) as inputs, and the ground truth labels as outputs,
Training the network weights of the feedback system, considering the input from the predicted output of the forward system's decoder network, and the ground truth label as outputs, and,
Training the network weights of the forward's system decoder part only, taking the inputs from previously extracted high-level features from the raw input image and the feedback ht from the feedback system. Here, the forward's system and the feedback system's encoder are designed to predict from previously learned and updated weights during the previous steps,
Repeat until convergence is reached.

The model has been preliminarily trained on a learning database comprising a plurality of MIP images calculated from 3D images data and, for each MIP image, a mask of the cancerous lesions derived from the tumor delineation of the 3D images by experts. The model can in particular be trained on a learning database comprising MIP images corresponding to sagittal and coronal maximum intensity projections of 3D imaging data and their corresponding lesion masks. In this case, the sagittal and coronal MIP images are treated independently, meaning that a single model is trained to transform either a coronal or sagittal MIP image as input into its corresponding mask.

Once a cancer lesion mask is extracted from a MIP image, said mask can be further processed or analyzed in order to compute at least one biomarker, for instance a prognosis indicator of survival of the patient or of progression-free survival of the patients.

In embodiments, the further processing 400 of the lesion mask may comprise computing an indicator of lesion dissemination I_{D}. Said indicator may be computed by estimating the largest distance between the lesion pixels belonging to the lesion mask, which may be implemented by computing the distance between pixels belonging to the lesion mask that are the farthest away according to two orthogonal axes and summing said distances.

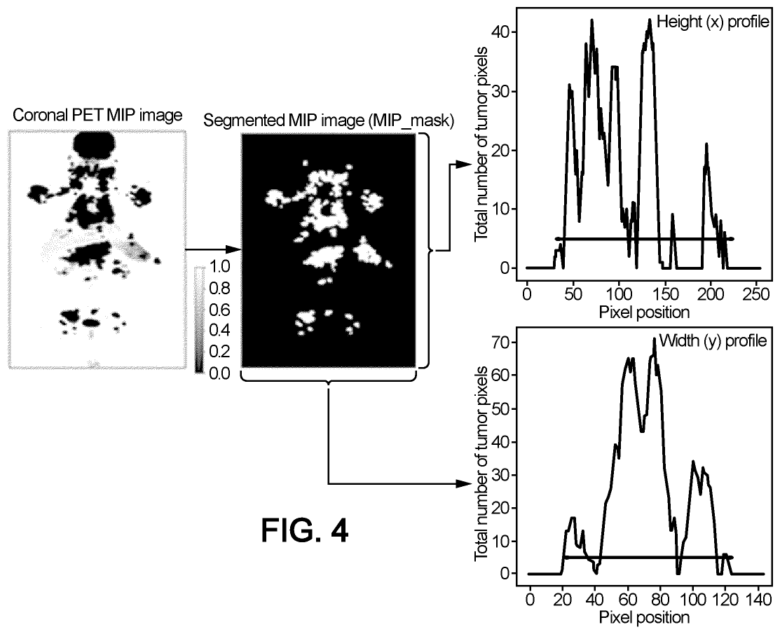

According to an embodiment schematically shown in FIG. 4, the computation of lesion dissemination may comprise calculating the sum of the pixels values (i.e. the sum of the pixels corresponding to the lesions since they are set to 1 and the other are set to 0) along the rows and columns of the lesion mask, yielding x and y profiles where the value of the profile for a line (y profile) or a column (x profile) is the number of pixels belonging to a lesion along the considered line or column.

In each profile, the largest distance is computed between a column, respectively line, corresponding to a given percentile a and a column, respectively line, corresponding to the percentile equal to 100−a, with a preferably between 0 and 10, preferably inferior to 5, for instance a=2. Pixel positions with zero total number of tumor pixels (often at the beginning and end of the pixel positions) are not considered for the percentile calculation.

The indicator of lesion dissemination may thus be computed, for a given MIP image and when setting a to 2, as I_{D}=(x_{98%}−x_{2%})+(y_{98%}−y_{2%})

When, for a patient, a MIP coronal image and a MIP sagittal image are calculated and corresponding lesion masks are obtained, the indicator of lesion dissemination is the sum of the indicators computed on each image:

$${I}_{D} = {I}_{D , coronal} + {I}_{D , sagittal}$$

In FIG. 4 is shown an example displaying the distances between the 2% percentile and the 98% percentile in x and y.

The further processing of the lesion mask may also, or alternatively, comprise the computation of an indicator of tumor burden, I_{B}, by computing a number of pixels belonging to the lesion, multiplied by the area represented by each pixel.

When, for a patient, a MIP coronal image and a MIP sagittal image are calculated and corresponding lesion masks are obtained, the indicator of lesion burden is the sum of the indicators computed on each image:

$${I}_{B} = {I}_{B , coronal} + {I}_{B , sagittal}$$

### Example—Materials and Methods

### Patients

The study population included DLBCL patients who had a baseline (before treatment initiation) PET/CT scan from two independent trials: REMARC (NCT01122472) and LNH073B (NCT00498043). PFS and OS as defined following the revised National Cancer Institute criteria were recorded. All data were anonymized before analysis. The institutional review board approval, including ancillary studies, was obtained for the two trials, and all patients provided written informed consent. The demographics and staging of the patients used for the survival analysis are summarized in Table 1.

TABLE 1

| Characteristic | REMARC | LNH073B |
|---|---|---|
| No. of patients | 287 | 95 |

Sex

| | | | | |
|---|---|---|---|---|
| No. of men | 165 | (57.5%) | 42 | (44%) |
| No. of women | 122 | (42.5%) | 53 | (56%) |
| Median age (y) | 68 | [64.0-73.0] | 46 | [33.25-55.0] |
| Median weight (kg) | 72 | [63.0-84.2] | 68 | [58.0-80.0] |
| Median height (cm) | 167.5 | [160.0-175.0] | 173 | [140.0-193.0] |

(1 case missed)

Ann Arbor stage

| | | | | |
|---|---|---|---|---|
| <I | 1 | (0.4%) | 0 | (0%) |
| >=II | 286 | (99.6%) | 95 | (100%) |

Performance status

| | | | | |
|---|---|---|---|---|
| 0 | 115 | (40%) | 0 | (0%) |
| 1 | 121 | (42%) | 27 | (28.4%) |
| 2 | 42 | (14.6%) | 43 | (45.3%) |
| 3 | 2 | (0.7%) | 20 | (21.1%) |
| 4 | 2 | (0.7%) | 5 | (5.3%) |

| | | | |
|---|---|---|---|
| Missing | 5 | (1.7%) | NA |

Note:
data in brackets are interquartile ranges (quartile one to quartile three).

### Measurement of Reference TMTV and Dmax

For the REMARC cohort, the lymphoma regions were identified in the 3D PET images as described in the following publications:
Vercellino L, Cottereau A S, Casasnovas O, et al. High total metabolic tumor volume at baseline predicts survival independent of response to therapy. Blood. 2020;135:1396-1405.
Capobianco N, Meignan M, Cottereau A -S, et al. Deep-Learning 18 F-FDG Uptake Classification Enables Total Metabolic Tumor Volume Estimation in Diffuse Large B-Cell Lymphoma. J Nucl Med. 2021;62:30-36.

A SUVmax 41% threshold segmentation was then applied on these regions, corresponding to including in the final region all voxels whose intensity was greater than or equal to 41% of the maximum intensity in the region.

The LNH073B lesions were segmented by first automatically detecting hypermetabolic regions by selecting all voxels with an SUV greater than 2 included in a region greater than 2 mL, and a 41% SUVmax thresholding of the resulting regions was used, corresponding to including in the final region all voxels whose intensity was greater than or equal to 41% of the maximum intensity in the region.

In all cohorts, physicians removed the regions corresponding to physiological uptakes and added pathological regions missed by the algorithm. The physicians were blinded to the patient outcomes. Expert-validated 3D lymphoma regions were used to compute the reference TMTV and Dmax (based on the centroid of the lymphoma regions).

### Calculation of the PET MIP Images and 2D Reference Lymphoma Regions

For each patient whole-body 3D 18F-FDG PET images and associated 3D lymphoma regions, two 2D MIP views and associated 2D lymphoma regions were calculated (FIG. 2). The 3D PET image was projected in the coronal and sagittal directions, 90° apart (FIG. 2), setting each pixel value of the projection to the maximum intensity observed along the ray normal to the plane of projection. Similarly, MIP of the expert-validated 3D lymphoma regions were calculated, resulting in binary images of 2D lymphoma regions (FIG. 2), hereafter called MIP_masks. These MIP_masks were then used as a reference output to train a CNN-based fully automatic lymphoma segmentation method.

### Fully Automatic Lymphoma Segmentation on PET MIP Images

To automatically segment the lymphoma lesions from the sagittal and coronal PET MIP images, a deep learning model was implemented.

The model consists of an encoder and a decoder network with a skipped connection between the two paths and external fully connected network-based feedback with a residual CNN as a building block (FIG. 3). The input and output dimensions of the network were 128×256×1.

The building block is the convolutional building block of the deep learning model. Each 2D CNN (Conv2D) with a kernel size of 3×3 was followed by batch normalization and activation function. The exponential linear unit (ELU) activation function was used, except it was a sigmoid activation function at the output layers. After the convolutional building block in the encoder, a 2×2 max pooling operation was applied, with stride 2 for downsampling. Before the convolutional building block, a 2×2 up-convolutional layer was used in the decoder.

All available 3D PET images and the corresponding expert-validated 3D lymphoma segmented regions were resized in to 4×4×4 mm3 voxel size. The resized 3D images were then padded or cropped to fit into a 128×128×256. The resized and cropped image were projected into sagittal and coronal views. The input and output image dimensions to the network were 128×256×1.

The sagittal and coronal PET MIPs were independent input images during training. The corresponding MIP_mask was the output image. The deep learning model was trained to transform a given sagittal or coronal PET MIP image to the corresponding MIP_mask with pixels of lymphoma regions set to one and pixels of the non-lymphoma regions set to zero.

First, using the REMARC cohort (298 patients), a five-fold cross-validation technique was used to train and evaluate the model. Patients were randomly split into five groups, and then five models were trained on 80% of the population and the remaining 20% was used for validation.

The model was trained with a batch size of 32 for 1000 epochs and 300 early stop criteria. The deep learning model neural network weights were updated using a stochastic gradient descent algorithm, ADAM optimizer, with a learning rate of 1e⁻⁴. All other parameters were Keras default values. A sigmoid output activation function was used to binarize the image into the lymphoma region and non-lymphoma region. The average of the Dice similarity coefficient (Loss_{Dice}) and binary cross-entropy (Loss_{binary cross-entropy}) was used as a loss function defined by:

$$loss = \frac{1}{2} ( {Loss}_{binary cross - entropy} + {Loss}_{Dice} )$$

The model was implemented with Python, Keras API, and Tensorflow backend. The data was processed using the Python 3.8.5 package, including Numpy, Scipy, Pandas, and Matplotlib. No post-processing method was applied for the segmentation metrics. To compute the surrogate biomarkers from the AI-based segmented images, regions with less than 4.8 cm2 were removed.

Secondly, the model trained from the REMARC cohort (298 patients) was tested on the independent LNH073B cohort (174 patients) to characterize its generalizability and robustness. The REMARC and LNH073B cohorts were acquired from two different trials. The REMARC (training-validation) data was a double-blind, international, multicenter, randomized phase III study, which started inclusion in 2010. In contrast, the LNH073B data was a prospective multicenter, randomized phase II study, that started inclusion including patients in 2007.

Calculation of I_{B} and I_{D}

Burden indicator I_{B} and Dissemination indicator I_{D}, interpreted respectively as surrogate indicators for TMTV and Dmax, were defined and computed from the MIP_masks automatically segmented from the coronal and sagittal PET MIP images using the deep learning method.

To characterize tumor burden I_{B}, the number of pixels belonging to the tumor regions in MIP_mask multiplied by the pixel area was computed. For a given patient, I_{B} was calculated from the coronal and the sagittal MIP_masks as I_{B}=I_{B,coronal}+I_{B,sagittal}. The dissemination of the disease I_{D} was analyzed by estimating the largest distance between the tumor pixels belonging to the MIP_mask. First, the sums of pixels along the columns and the rows of MIP_mask were calculated, yielding x and y profiles (FIG. 4). Second, in each of these two profiles, the distances between the 2% percentile and the 98% percentiles (x_{2%} and x_{98%} in the x profiles, y_{2%} and y_{98%} in the y profiles) were calculated, yielding (x_{98%}−x_{2%}) and (y_{98%}−y_{2%}), respectively. These percentiles were chosen to improve the robustness of the calculation to outliers. The largest distance was defined as

$${I}_{D} = ( {x}_{98 \%} - {x}_{2 \%} ) + ( {y}_{98 \%} - {y}_{2 \%} )$$

For a given patient, the tumor dissemination I_{D} was the sum of the coronal and sagittal disseminations using

$${I}_{D} = {I}_{D , coronal} + {I}_{D , sagittal}$$

### Statistical Analysis

Using the MIP_masks obtained from the expert-delineated 3D lymphoma regions (FIG. 2) as a reference, CNN's segmentation performance was evaluated using the Dice score, sensitivity, and specificity. The difference between the CNN-based segmentation results and the expert-delineated 3D lymphoma regions were quantified using Wilcoxon statistical tests. Univariate and multivariate survival analyses were performed. For all biomarkers, a time-dependent area under the receiver operating characteristics curve (AUC) was calculated. Bootstrap resampling analysis was performed to associate confidence intervals to the Cox model hazard ratio and the time-dependent AUC. Test results were considered statistically significant if the two-sided P-value was <0.05.

### RESULTS

A total of 475 patients from two different cohorts were included in this study, of which 93 patients were excluded from the biomarker and survival analysis because the provided baseline 18F-FDG PET/CT images were not suitable to analyze all biomarkers (no PET segmentation by an expert or less than 2 lesions).

The performance of the proposed segmentation method was evaluated patient-wise. The CNN segmentation method achieved a 0.80 median Dice score (interquartile range [IQR]: 0.63-0.89), 80.7% (IQR: 64.5%-91.3%) sensitivity, and 99.7% (IQR: 99.4%-0.99.9%) specificity on the REMARC cohort. On the testing 174 LNH073B patients, the CNN yielded a 0.86 median Dice score (IQR: 0.77-0.92), 87.9% (IQR: 74.9.0%-94.4%) sensitivity, and 99.7% (IQR: 99.4%-99.8%) specificity. In the LNH073B data, the CNN yielded a mean Dice score of 0.80±0.17 (mean±SD) on the coronal view and 0.79±0.17 on the sagittal view. FIG. 2 shows segmentation result examples from experts (MIP_masks) and CNN. The Dice score was not significantly different (p>0.05) between the coronal and sagittal views, both for the REMARC and LNH073B cohorts (p>0.05).

In both cohorts, there was a significant correlation between ranked TMTV and Dmax values and the associated surrogate values I_{B}, I_{D} obtained using CNN. For REMARC, TMTV was correlated with I_{B} (Spearman r=0.878, p<0.001), and Dmax was correlated with I_{D} (r=0.709, p<0.001). Out of 144 patients who had TMTV greater than the median TMTV (242 cm3), 121 (84.02%) patients had also I_{B} greater than the median I_{B} (174.24 cm2). 144 patients had Dmax greater than the median Dmax (44.8 cm), and 113 (78.5%) of these patients also had I_{D} greater than the median I_{D} (98.0 cm).

For LNH073B, TMTV was correlated with I_{B} (r=0.752, p<0.001), and Dmax was correlated with I_{D} (r=0.714, p<0.001). Out of 48 patients who had TMTV greater than the median TMTV (375 cm3), 42 (87.5%) patients had also I_{B} greater than the median I_{B} (307.2 cm2). 48 patients had Dmax greater than the median Dmax (44.1 cm), and 39 (81.3%) of these patients also had I_{D} greater than the median I_{D} (116.4 cm). Table 2 shows the descriptive statistics for the I_{B} and I_{D}.

TABLE 2

| | | | | | Q1 | | Q3 | |
|---|---|---|---|---|---|---|---|---|
| Cohort | I_{B}/I_{D} | Mean | SD | Minimum | (25%) | Median | (75%) | Maximum |
| REMARC | I_{B} | 252.27 | 245.75 | 0.48 | 77.04 | 174.24 | 350.56 | 1339.36 |
| | (cm²) | | | | | | | |
| | I_{D} | 100.16 | 49.89 | 0.40 | 66.20 | 98.0 | 135.0 | 225.20 |
| | (cm) | | | | | | | |
| LNH073B | I_{B} | 388.12 | 249.91 | 63.68 | 224.48 | 307.2 | 450.08 | 1186.24 |
| | (cm²) | | | | | | | |
| | I_{D} | 121.82 | 41.10 | 43.20 | 92.00 | 116.40 | 145.60 | 222.40 |
| | (cm) | | | | | | | |

### Survival Analysis

The time-dependent AUC and hazard ratios (HR) with 95% confidence interval of the metabolic tumor volume and tumor spread are shown in Table 3 for the REMARC and LNH073B data. All PET features extracted from the baseline 3D 18F-FDG PET/CT images and using AI (I_{B} and I_{D}) were significant prognosticators of the PFS and OS.

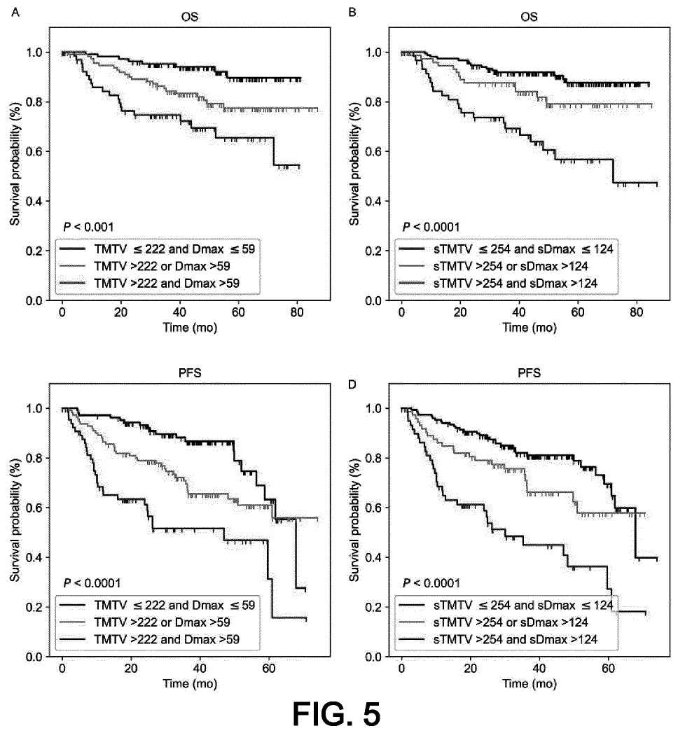

Combining TMTV and Dmax (or their surrogates), three risk categories could be differentiated in the REMARC data (FIG. 5): using the 3D features, category 1 corresponded to low TMTV (≤222 cm3) and low Dmax (≤59 cm) (low risk, n=108); category 2 corresponded to either high Dmax or high TMTV (intermediate risk, n=112); category 3 corresponded to both high Dmax and high TMTV (high risk, n=67). This stratification was similar when using the MIP-features-based categories using AI (FIG. 5). The accuracy of the CNN-based classification into three categories with respect to the 3D-biomarkers-based classification was 71.4%.

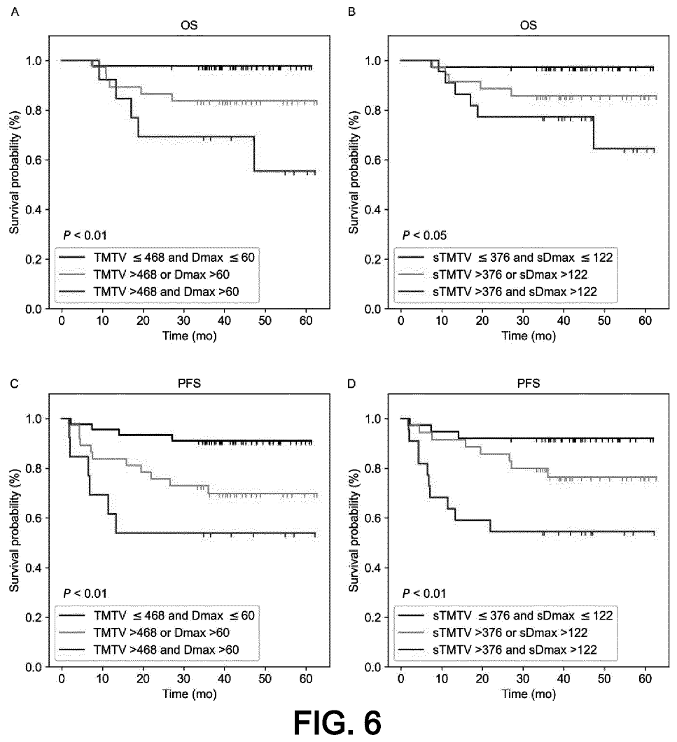

In the LNH073B cohort, combining TMTV and Dmax (or their surrogates), three risk categories could be differentiated (FIG. 6). Using the 3D features, category 1 was defined as low TMTV (≤468 cm3) and low Dmax (≤60 cm) (n=45); category 2 corresponded to either high Dmax or high TMTV (n=37); category 3 corresponded to both high Dmax and high TMTV (n=13). Out of the 13 patients classified as high risk, 9 (69.2%) patients had less than 4-years of OS, and 10 (76.9%) patients had less than 4-years of PFS. This stratification was similar when using the CNN-based results. The I_{B} cut-off value was 376 cm2, the I_{D} cut-off value was 122 cm. There were 38 patients in category 1, 35 in category 2, and 22 in category 3. Out of the 22 patients classified as a high risk, 19 (77.3%) patients had less than 4-years of OS, and 19 (86.4%) patients had less than 4-years of PFS. The accuracy of the AI-based classification into three categories with respect to the 3D-biomarkers-based classification was 64.2%. All patients classified as high risk using the 3D biomarkers were also classified as high risk using the CNN, except one patient who had an OS of 36.6 months. Out of the nine patients classified as high risk when using the CNN but not when using the 3D biomarkers, 8 (88.9%) patients had less than 4-years of OS, and the remaining one (11.1%) patient had 21.95 and 57.99 months of PFS and OS respectively.

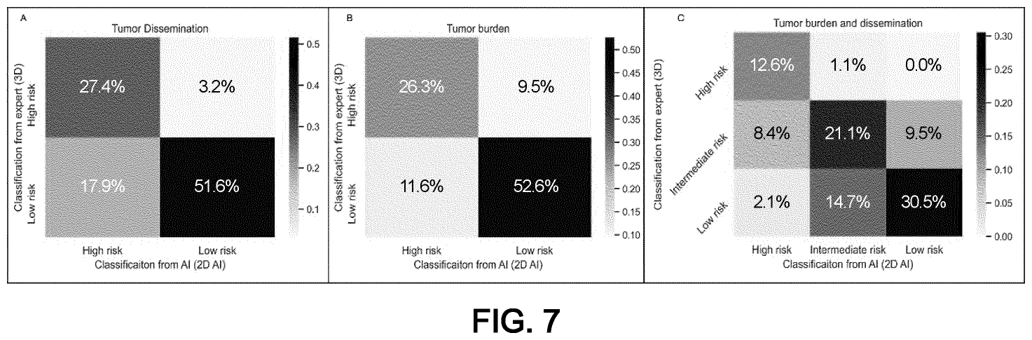

In FIG. 7, the confusion matrices show the agreement between the 3D-based biomarkers and the surrogate MIP biomarkers in the LNH073B data. The percentage of the data classified into high, low, and intermediate risk is also shown. Considering one biomarker-based classification, the AI-based classification into two groups (high and low risks) to the 3D-based classification (using either tumor burden or dissemination biomarkers) was 79% accuracy.

| | | | 3D ¹⁸F-FDG PET/CT estimates | | 2D PET MIP estimates | |
|---|---|---|---|---|---|---|
| Data | PFS/OS | Metrics | TMTV | Dmax | I_{B} | I_{D} |
| REMARC | PFS | AUC | 0.67 | 0.65 | 0.65 | 0.68 |
| | | | (0.60-0.73) | (0.58-0.72) | (0.58-0.72) | (0.62-0.75) |
| | | HR | 11.24 | 9.0 | 11.81 | 12.49 |
| | | | (2.10-46.20) | (2.53-23.63) | (3.29-31.77) | (3.42-34.50) |
| | OS | AUC | 0.67 | 0.62 | 0.67 | 0.68 |
| | | | (0.58-0.76) | (0.53-0.71) | (0.58-0.76) | (0.59-0.76) |
| | | HR | 16.43 | 8.60 | 22.14 | 22.79 |
| | | | (2.42-77.29) | (1.47-28.33) | (4.73-69.06) | (3.80-79.21) |
| LNH073B | PFS | AUC | 0.62 | 0.56 | 0.66 | 0.58 |
| | | | (0.49-0.75) | (0.39-0.72) | (0.53-0.80) | (0.41-0.74) |
| | | HR | 13.79 | 32.83 | 9.24 | 16.79 |
| | | | (0.45-86.80) | (0.4-220.8) | (0.95-37.94) | (0.69-86.41) |
| | OS | AUC | 0.65 | 0.51 | 0.64 | 0.50 |
| | | | (0.46-0.82) | (0.31-0.72) | (0.45-0.82) | (0.29-0.72) |
| | | HR | 64.30 | 49.21 | 14.17 | 20.39 |
| | | | (0.74-384.80) | (0.07-258.3) | (0.59-67.02) | (0.08-93.66) |

Thus, the automated segmentation of lesion mask of Maximum Intensity Projection Images obtained from 3D imaging data provides accurate and less computationaly intensive segmentation, and the obtained lesion masks can provide prognostic indicators reflecting tumor burden and tumor dissemination.

1. A method of processing imaging data of a patient having cancer, comprising:
providing three-dimensional imaging data of the patient,
computing from said three-dimensional imaging data, at least one two-dimensional Maximum Intensity Projection (MIP) image, corresponding to a projection of the maximum intensity of the three-dimensional imaging data along one direction onto one plane,
extracting a mask of the MIP image corresponding to cancerous lesions by application of a trained model.

2. The method according to claim 1, wherein the three-dimensional imaging data is PET scan data.

3. The method according to claim 1, comprising computing from the three-dimensional imaging data two MIP images corresponding to the projection of the maximum intensity of the three-dimensional imaging data onto two orthogonal planes.

4. The method according to claim 3, wherein the trained model has been previously trained by supervised learning on a database comprising a plurality of MIP images corresponding to projections of three-dimensional imaging data according to a first plane, and a plurality of MIP images corresponding to projections of three-dimensional imaging data according to a second plane, orthogonal to the first, and, for each MIP image, a corresponding mask of the image corresponding to cancerous lesions.

5. The method according to claim 1, wherein the trained model is a Convolutional Neural Network comprising a forward system comprising:
an encoder region comprising a succession of layers of decreasing resolutions,
a decoder region comprising a succession of layers of increasing resolutions, wherein a layer of the decoder region concatenates the output of the layer of the encoder region of the same resolution with the output of the layer of the decoder region of the next lower resolution,
a bottle-neck region between the encoder and decoder regions,
and a feedback system, comprising an encoder part and decoder part respectively identical to the encoder region and the decoder region of the forward system, where the output of the encoder part is concatenated to the output of the layer of lowest resolution of the forward system for at least one training phase of the network.

6. A method according to claim 5, wherein the encoder, decoder and bottle-neck regions of the network comprise building blocks where each building block is a residual block comprising at least a convolutional layer and an activation layer, with a skip connection between the input of the block and the activation layer.

7. A method for assisting with cancer prognosis comprising:
performing the method according to claim 1 on three-dimensional imaging data of a patient to output a two-dimensional cancerous lesion mask of a MIP image computed from the three-dimensional imaging data, and
processing said cancerous lesion mask to compute at least one prognosis indicator.

8. The method according to claim 7, wherein the at least one prognosis indicator comprises an indicator of the lesion dissemination.

9. The method according to claim 8, wherein processing the cancerous lesion mask comprises computing the distance between tumor pixels belonging to the cancerous lesion mask along two orthogonal axes of the cancerous lesion mask and summing said dimensions.

10. The method according to claim 7, wherein the at least one prognosis indicator comprises an indicator of the lesion burden.

11. The method according to claim 10, wherein processing the cancerous lesion mask comprises computing a number of pixels belonging to the lesion multiplied by the area represented by each pixel.

12. The method according to claim 7, wherein the cancer is a lymphoma.

13. The method according to claim 12, wherein the lymphoma is Diffuse Large B-cell Lymphoma.

14. (canceled)

15. A non-transitory computer readable storage having stored thereon code instructions for implementing the method according to claim 7, when they are executed by a processor.

16. A non-transitory computer readable storage having stored thereon code instructions for implementing the method according to claim 1, when they are executed by a processor.
